(19) United States
(12) Patent Application Publication
(10) Pub. No.: US 2015/0290031 A1
(43) Pub. Date: Oct. 15, 2015

(54) TOUCHLESS USER INTERFACE FOR OPHTHALMIC DEVICES

(71) Applicant: Novartis AG, Basel (CH)

(72) Inventor: Armin Wellhoefer, Schwaig (CH)

(73) Assignee: Wavelight GmbH, Erlangen (DE)

(21) Appl. No.: 14/389,341

(22) PCT Filed: May 16, 2013
(86) PCT No.: PCT/EP2013/060157

§ 371 Date: Sep. 29, 2014

(51) Int. Cl. A61F 9/008; A61B 19/00; G06F 3/03; G06F 3/01; G06F 3/16

(52) U.S. Cl. CPC A61F 9/008; G06F 3/017; G06F 3/167; G06F 3/03; A61B 19/56

(57) ABSTRACT

An ophthalmic apparatus for laser eye surgery comprising a command recognition unit configured for detecting and recognizing a gesture command and/or voice command of an operator of the ophthalmic apparatus, at least one controlled unit configured for receiving a control signal and configured for changing a state based on the received control signal, and a controller configured for generating a control signal and transmitting the control signal to the at least one controlled unit based on the recognized gesture command and/or voice command.

This invention relates to a touchless user interface for ophthalmic devices, and in particular to an ophthalmic apparatus capable of recognizing a gesture command and/or voice command for controlling at least one unit of the ophthalmic apparatus.

### BACKGROUND OF THE INVENTION

In the fields of ophthalmic surgery, ophthalmic treatment and ophthalmic diagnosis, devices are employed which include a variety of components and units controlled by a user of the devices. Conventionally this control takes place via a user interface, such as a keyboard, a touchscreen, a joystick or the like. Before a surgery takes place, the operator, for example an ophthalmologist, sterilizes the hands and puts on sterile cloth and gloves, in order to protect the patient from an infection.

Since the ophthalmologist has to touch the user interface to operate and control the device, the device itself needs to be sterilized as well. For instance, for each surgery the device can be cleaned and/or covered with a sterile transparent foil, which is removed after the surgery. However, such sterile cover obstructs the view to the device and, and in particular its user interface.

### SUBJECT OF THE INVENTION

It is therefore an object of the invention to provide an ophthalmic apparatus which can be operated in an easy manner, while being in a sterilized environment.

This object is solved by the present invention as claimed in the independent claim. Preferred embodiments are defined by the dependent claims.

In accordance with an aspect of the present invention, an ophthalmic apparatus for laser eye surgery is provided which comprises a command recognition unit configured for detecting and recognizing a gesture command and/or voice command of a user of the ophthalmic apparatus. The apparatus further includes at least one controlled unit configured for receiving a control signal and configured for changing a state based on the received control signal, and a controller configured for generating a control signal and transmitting the control signal to the at least one controlled unit based on the recognized gesture command and/or voice command. Such an ophthalmic apparatus provides the advantage that its surface does not meet to be sterilized for a laser eye surgery, since the operator must not touch the surface of the apparatus.

According to a further aspect, the ophthalmic apparatus may further comprise a memory configured for storing one or more commands in association with gesture data and/or voice data.

According to yet another aspect of the present invention, the command recognition unit may comprise a detection unit configured for detecting a gesture and/or voice of the operator of the ophthalmic apparatus, an evaluation unit configured for evaluating the detected gesture and/or voice and generating gesture data and/or voice data respectively representing the evaluated gesture and/or voice, and a determination unit configured for determining a command associated with the gesture data and/or voice data. Such command recognition unit is capable of identifying one or more commands for controlling the controlled unit(s) in a very user convenient manner, since the user must not release any instrument from his/her hands to perform control of the ophthalmic apparatus.

In accordance with an aspect of the invention, the detection unit is coupled to at least one of a camera, a motion sensor, a microphone, an infrared detector, a radio frequency identification (RFID) detector, a Bluetooth transceiver, a Global Positioning System (GPS) and a Differential Global Positioning System (DGPS).

In accordance with a further aspect, the at least one controlled unit may include at least one of a laser unit, a microscope, and a part or an entire bed for a patient of the laser eye surgery.

According to another aspect of the present invention, the ophthalmic apparatus may further comprise a footswitch configure for activating the command recognition unit and/or the controller.

According to yet another aspect of the present invention, the ophthalmic apparatus may further comprise a security unit configured for identifying the operator of the ophthalmic apparatus based on an utterance made by the operator, a form of a body part of the operator and/or a wearable object worn by the operator.

In accordance with an aspect of the invention, the memory is further configured for storing a linguistic profile, a voice profile, a body part profiles and/or one or more wearable object identifiers in association with each operator of the ophthalmic apparatus, and the security unit is configured for determining an operator based on a comparison of the utterance made by the operator, the form of the body part of the operator and/or the wearable object worn by the operator with the stored profiles and/or identifiers.

### BRIEF DESCRIPTION OF THE DRAWINGS

The invention is explained below in more detail on the basis of the attached drawings, of which:

FIG. 1 schematically illustrates components and units of an ophthalmic apparatus according to an embodiment, and

FIG. 2 schematically illustrates further elements of the ophthalmic apparatus, which can be included or coupled to a command recognition unit according to an embodiment.

### DETAILED DESCRIPTION OF THE DRAWINGS

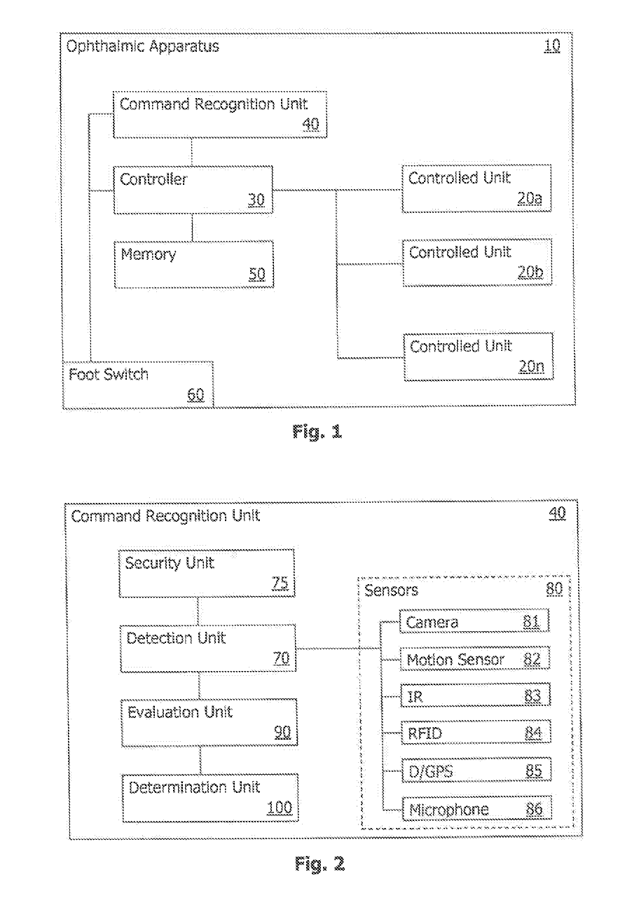

FIG. 1 illustrates a schematic view of an ophthalmic apparatus in accordance with an embodiment of the present invention. The ophthalmic apparatus is any kind of device for an ophthalmologic surgery, treatment and/or diagnosis. For example, the ophthalmic apparatus may be a femtosecond laser (FS laser) device, an excimer laser (EX laser) device, a device forming a combination of an FS- and EX-laser device or any other device employed during an eye surgery or treatment, such as a LASIK treatment (LASIK: Laser in-situ keratomileusis).

The ophthalmic apparatus 10 includes at least one controlled unit 20. According to FIG. 1 a plurality of controlled units indicated by the reference numerals 20a, 20b to 20n, herein referred to as controlled unit 20, are depicted. However, the present invention is not restricted to the number of controlled units illustrated in the Figures but rather comprises any number of controlled units necessary for the surgery or treatment.

A controlled unit 20 is a component of the ophthalmic apparatus 10 that can be controlled by the operator. According to this embodiment controlling includes moving, altering, fine-tuning the controlled unit 20 with an actuator (not shown) or setting an adjustable parameter by the operator. Examples of controlled units 20 are a power unit, a laser source, light source, focusing optics, scanning components, microscopic devices, measuring devices (e.g., pachymeter), head-up display, an examination table or bed including a head part, a body part and a foot rest on which the patient lies or sits, etc. A further controlled unit can be a patient administration program or parts thereof, such as menus.

Thus, a controlled unit refers to any component of the ophthalmic apparatus which can be moved, steered, tuned, switched on and off and/or has a parameter value to be set by the operator.

The controlled units 20 are coupled to a controller 30 via, for example, a bus system or bus interface of the ophthalmic apparatus. The controller 30 generates a control signal for each of the controlled units 20, such as a signal for actuating a motor or other actuator, switching on and off a power source of the ophthalmic apparatus and/or an individual power source of a controlled unit, switching the controlled unit from one state to another, setting a particular parameter, such as the intensity of a laser radiation, the sensibility of a sensor, etc.

In accordance with the present invention, the ophthalmic apparatus further includes a command recognition unit 40, which detects and recognizes a gesture command and/or a voice command of an operator of the ophthalmic apparatus. A gesture command is any gesture, i.e. motion of a hand, arm, head, eye or any other parts of the body of the operator, indicating a particular control command for controlling the ophthalmic apparatus and its components. For instance, the operator may perform a particular gesture with his or her fingers, which is detected by the command recognition unit 40 and recognized as a particular gesture corresponding to a particular operation of a controlled unit 20. Further, a voice command is any utterance, such as a sound, a word or even a spoken sentence rendered or uttered by the operator of the ophthalmic apparatus. The command recognition unit 40 recognizes it as a particular voice command corresponding to an operation of a controlled unit 20.

The command recognition unit 40 is not limited to recognizing a gesture command and/or a voice command. It can also recognize a combination of gesture and voice. For instance, the operator can move his/her hand in a certain manner and say “ON” or “OFF”. The command recognition unit 40 is capable of detecting both commands as a combined command for switching on or off a particular controlled unit 20 associated with the gesture.

When the command recognition unit 40 has detected and recognized a gesture command and/or voice command and/or combined command, it sends a corresponding signal to the controller 30. The controller 30 then generates a control signal and transmits the control signal to at least one controlled unit 20 to perform the operation of the controlled unit 20 as desired by the operator. As an example only, the operator can make a particular gesture or can say one or more words to move a laser unit and make another gesture and/or utterance to move the head rest of the apparatus. Further commands can move the laser source, move the optics, change the intensity of the laser, etc.

In order to correctly generate control signals associated with recognized gesture commands and/or voice commands the ophthalmic apparatus provides a memory 50. The memory 50 stores command data in association with gesture data and/or voice data. Command data can be any indication of a particular control command designated for at least one controlled unit 20. For example, such command data represents the movement of a movable controlled unit 20, represents switching a switchable controlled unit 20, or represents the adjustment of a certain parameter of a parameterizable controlled unit 20.

Each of the commands represented by the command data is associated with one or more gesture data and/or voice data. This gesture and/or voice data is either sensor data captured by a gesture or voice sensor, or data resulting from a calculation process performed by the command recognition unit. For instance, the command recognition unit may detect a gesture and/or voice received by a sensor (which will be explained further below with reference to FIG. 2) and perform certain calculations or processing on the detected gesture and/or voice to generate gesture data and/or voice data. The latter may exemplarily comprise quantized data of a recognized movement of the operator or quantized voice data.

The memory 50 therefore includes data sets, where particular gesture data and/or voice data is associated with a particular command for operating the controlled units 20. To allow accurate command recognition, particular gestures and/or voice can be trained for each command available for the controlled units 20 of the ophthalmic apparatus 10. The memory 50 then stores one or more data sets for each command to allow varying gestures or utterances to be associated with the same command. Memory 50 can also store various data sets for different operators (users), so that individual gestures and/or utterances can be associated with the possible commands for the controlled units 20.

As shown in FIG. 1, the ophthalmic apparatus 10 further includes a switch 60, which could be a foot switch, a sensor barrier or any other type of switch, which can be operated without using the hands or other sterile parts of the operator. The switch 60 is configured to activate or deactivate the controller 30 and/or the command recognition unit 40. Thus, the command recognition and controlling of the ophthalmic apparatus 10 can only be performed if the switch 60 is switched on. For example, the operator, such as an ophthalmologist, may first activate a foot switch before making a hand gesture or before uttering a command.

It is now referred to FIG. 2, illustrating in more detail the command recognition unit 40 of FIG. 1.

The command recognition unit 40 may include a detection unit capable of detecting a gesture and/or voice of the operator. In order to achieve this detection, the command recognition unit further includes one or more sensors 80. It is to be understood by those skilled in the art, that the sensors 80 are not necessarily part of the command recognition unit 40, but can be connected, i.e. electrically and/or electronically coupled, to the ophthalmic apparatus 10 and/or command recognition unit 40.

The sensors 80 may be any suitable sensor, such as a camera 81, a motion sensor 82, an infrared sensor 83, a RFID sensor 84, a GPS or DGPS sensor 85 as well as a microphone 86. The present invention is not limited to these sensors but can comprise any other sensor capable of sensing a touchless control operation.

According to an example, detection could be accomplished by an infrared light that is transmitted in the direction of the operator. A reflection of the infrared light can be received by a camera 81 or IR sensor 83, so that the distance of a body part of the operator as well as a direction vector or vectors of a movement can be retrieved. Instead of infrared sensors 83 other motion sensors 82 or even supersonic sensors (not shown), i.e. a supersonic source and supersonic receiver, can be used with the present invention. To improve capturing of a movement, more than one camera could be installed. In any case, the detection unit receives a signal from at least one of the sensors 80 and determines whether it is a gesture and/or voice of the operator.

In order to avoid misuse of the ophthalmic apparatus or control thereof by other people than the operator, the command recognition unit 40 can include a security unit 75. The security unit 75 is configured for identifying the operator of the ophthalmic apparatus based on an utterance made by the operator, a form of a body part of the operator and/or a wearable object worn by the operator. For instance, the detection unit 70 can pass a received sensor signal or signals, such as the signals described above, to the security unit 75.

The security unit 75 then compares an utterance made by the operator, the form of a body part of the operator and/or the wearable object worn by the operator based on the received signal(s) with one or more stored profiles and/or identifiers of objects. The memory 50 can store a linguistic profile, a voice profile, a body part profiles and/or one or more wearable object identifiers in association with each operator of the ophthalmic apparatus for such comparison. Thus, only if a received utterance matches a linguistic or voice profile, a received form of a body part matches a body part profile and/or if an identifier of a wearable object matches a stored identifier, the detection unit proceeds further. Otherwise, the received signal(s) is discarded.

A wearable object can be identified by an RFID-chip, a particular light source (e.g., an infra-red LED) or simply a certain color. For instance, each operator may wear gloves with a certain color different from the color of the gloves of other operators. The present disclosure, therefore, allows an easy and an inexpensive way of distinguishing between different operators.

Either after a successful security check or without any security measures, the received sensor signal or signals are then passed to an evaluation unit 90 which evaluates the gesture and/or voice. For instance, if a movement of a hand of the operator is captured by the camera 81 or another sensor 82, 83, the evaluation unit 90 performs image processing or sensor signal processing to evaluate the received sensor signals and to generate gesture data and/or voice data. This gesture and/or voice data represents each evaluated gesture and/or voice. The gesture data and/or voice data may include a quantization of movement vectors or quantization of received sound signals. Further, particular points of a movement or pitches within a voice can be evaluated and stored as gesture data and/or voice data characterizing the movement performed or the utterance spoken by the operator.

This characterizing gesture data and/or voice data is then compared by a determination unit 100 with already stored data, such as the trained gesture data and/or voice data stored in memory 50. If a match is determined, the determination unit 100 outputs a signal associated with the matching gesture data and/or voice data to the controller 30.

As a result, the command recognition unit 40 is capable of associating a command with a detected gesture and/or voice. Providing the determined command to the controller 30 allows an operation of the ophthalmic apparatus 10 without the necessity of the operator to use a button, touchscreen, joystick, or the like. Thus, the present invention provides a touchless operation of the ophthalmic apparatus 10. This avoids the conventional necessity of sterilization of the complete ophthalmic apparatus 10 or to cover the ophthalmic apparatus 10 with a sterilized transparent foil.

In accordance with a further embodiment of the present invention, the gesture recognition can be enhanced by providing a “data glove” or “data wrist band” which is worn by the operator. In more detail, the operator may wear a particular device which includes one or more transceiving modules. The transceiving modules can recognize their location information within particular time periods, such as a few milliseconds. Thus, a movement of wearable device and hence the operator can be detected. The current location information for each time period is then transmitted to a corresponding receiver at the ophthalmic apparatus 10. For instance, such a system could be implemented with an RFID system, where the RFID sensor 84 (see FIG. 2) activates one or more RFID chips provided in a glove or wrist band. These RFID chips then transmit location information determined within a predefined three-dimensional space. On the other hand, the one or more RFID chips can already detect and transmit movement information, for example, based on a gyroscopic sensor. A recognition and control system according to yet another embodiment of the present invention is based on a GPS system and/or a differential GPS system (DGPS system) and/or a Bluetooth system installed within the ophthalmologic apparatus.

Transmitters and receivers necessary for detecting a gesture, such as sensors 80, can be installed within an operation room for an ophthalmic surgery or treatment. The transmitters and receivers can then be installed in the direct vicinity of the operator to improve the accuracy of the gesture recognition. In this case, the receivers are coupled to the ophthalmic apparatus 10, such as to the command recognition unit 40, and more particularly to the detection unit 70, to allow command recognition in accordance with the present invention.

In accordance with yet another embodiment, the operator, such as the ophthalmologist, wears glasses comprising eye movement detectors. Such glasses detect a respective eye movement. The operator makes a gesture by looking to a particular point or moving one or both eyes in a certain manner. This gesture is then sensed by one or more sensors within the glasses and corresponding sensor signals are transmitted to the ophthalmic apparatus 10, i.e. command recognition unit 40 or detection unit 70.

The present invention has been described with respect to particular embodiments and examples. It is understood by those skilled in the art that combinations of these embodiments and examples also fall into the scope of the present invention.

1. An ophthalmic apparatus for laser eye surgery comprising:
a command recognition unit configured to detect and recognize a gesture command or voice command of an operator of the ophthalmic apparatus;
at least one controlled unit configured to receive a control signal and to change a state based on the received control signal; and
a controller configured to generate a control signal and to transmit the control signal to the at least one controlled unit based on the recognized gesture command or voice command.

2. The ophthalmic apparatus according to claim 1, further comprising:
a memory configured to store one or more commands in association with gesture data or voice data.

3. The ophthalmic apparatus according to claim 1, wherein the command recognition unit comprises:
a detection unit configured to detect a gesture or voice of the operator of the ophthalmic apparatus;
an evaluation unit configured to evaluate the detected gesture or voice and to generate gesture data or voice data representing the evaluated gesture or voice; and
a determination unit configured to determine a command associated with the gesture data or voice data.

4. The ophthalmic apparatus according to claim 3, wherein the detection unit is coupled to at least one of a camera, a motion sensor, a microphone, an infrared detector, a radio frequency identification detector, a Bluetooth transceiver, a GPS system and a DGPS system.

5. The ophthalmic apparatus according to claim 1, wherein the at least one controlled unit includes at least one of a laser unit, a microscope and a bed for a patient of the laser eye surgery.

6. The ophthalmic apparatus according to claim 1, further comprising:
a foot switch operable by the operator with a foot and configured to activate the command recognition unit or controller.

7. The ophthalmic apparatus according to claim 1, further comprising:
a security unit configured to identify the operator of the ophthalmic apparatus based on an utterance made by the operator, a form of a body part of the operator or a wearable object worn by the operator.

8. The ophthalmic apparatus according to claim 7, wherein the memory is further configured to store a linguistic profile, a voice profile, a body part profiles or one or more wearable object identifiers in association with each operator of the ophthalmic apparatus, and wherein the security unit is configured to determine an operator based on a comparison of the utterance made by the operator, the form of the body part of the operator or the wearable object worn by the operator with the stored profiles or identifiers.
